(19) United States
(12) Patent Application Publication
(10) Pub. No.: US 2020/0197416 A1
(43) Pub. Date: Jun. 25, 2020

(54) PHARMACEUTICAL COMPOSITION COMPRISING ABIRATERONE ACETATE AND DARULOTAMIDE

(71) Applicant: NanGenex Nanotechnology Incorporated, Msida (MT)

(72) Inventors: Andrea UJHELYI, Nyiregyhaza (HU); Nikoletta ERDOSI, Nyiregyhaza (HU); Betti SZABÓNÉ ORDASI, Budapest (HU); Tamás JORDÁN, Öcsöd (HU); Tamás SOLYMOSI, Békéscsaba (HU); Hristos GLAVINAS, Szeged (HU)

(21) Appl. No.: 16/637,206

(22) PCT Filed: Aug. 9, 2018
(86) PCT No.: PCT/US2018/046030

§ 371 Date: Feb. 6, 2020

Related U.S. Application Data
(60) Provisional application No. 62/543,023, filed on Aug. 9, 2017.

(51) Int. Cl. A61K 31/58; A61K 31/4155; A61K 9/00

(52) U.S. Cl. CPC A61K 31/58; A61K 9/0053; A61K 31/4155

(57) ABSTRACT

Disclosed herein are pharmaceutical compositions comprising Abiraterone acetate and Darolutamide, which are useful in the treatment of a type of prostate cancer. The pharmaceutical composition possesses increased in-vitro permeability both in fasted and fed state which allows significant dose reduction and the abandoning of the requirement of taking the drugs on an empty stomach. Further disclosed are methods of formulating and manufacturing the pharmaceutical composition, its uses and methods of treatment using the pharmaceutical composition.

This application claims the benefit of priority to US provisional application No. 62/543,023, filed Aug. 9, 2017, the disclosure of which is hereby incorporated by reference as if written herein in its entirety.

### FIELD OF THE INVENTION

Disclosed herein are pharmaceutical compositions comprising Abiraterone acetate and Darolutamide, which is useful in the treatment of prostate cancer. More specifically, the pharmaceutical composition possesses increased in-vitro permeability both in fasted and fed state which allows significant dose reduction and the abandoning of the requirement of taking the drugs on an empty stomach. Further disclosed are methods of formulating and manufacturing said pharmaceutical composition, its uses and methods of treatment using the pharmaceutical composition.

### BACKGROUND OF THE INVENTION

### Abiraterone

Abiraterone is a potent and selective inhibitor of CYP17 (17α-hydroxylase/C17,20-lyase). As Abiraterone was poorly bioavailable and also susceptible to hydrolysis by esterases, a prodrug was developed. Abiraterone acetate (A) was found to be resistant to esterases and was rapidly deacetylated to Abiraterone (B) in vivo, resulting in potent CYP17 inhibition. Abiraterone acetate is designated chemically as (3β)-17-(3-pyridinyl) androsta-5,16-dien-3-yl acetate and its structure is:

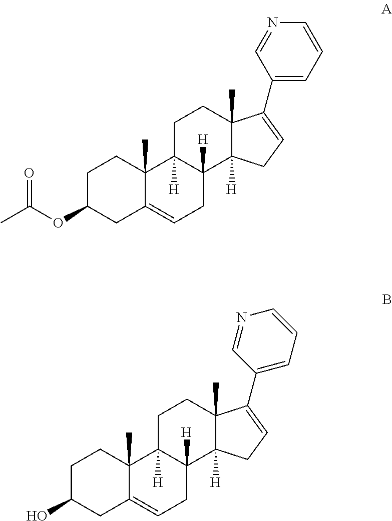

Abiraterone acetate is a white to off-white, non-hygroscopic, crystalline powder. Its molecular formula is C₂₆H₃₃NO₂ and it has a molecular weight of 391.55. Abiraterone acetate is a lipophilic compound with an octanol-water partition coefficient of 5.12 (Log P) and is practically insoluble in water. The pKa of the aromatic nitrogen is 5.19.

Inactive ingredients in the Zytiga® tablets are colloidal silicon dioxide, croscarmellose sodium, lactose monohydrate, magnesium stearate, microcrystalline cellulose, povidone, and sodium lauryl sulfate. Each Zytiga® tablet contains 250 mg of Abiraterone acetate.

Abiraterone acetate (ZYTIGA) is converted in vivo to Abiraterone, an androgen biosynthesis inhibitor, that inhibits 17α-hydroxylase/C17,20-lyase (CYP17). This enzyme is expressed in testicular, adrenal, and prostatic tumor tissues and is required for androgen biosynthesis.

CYP17 catalyzes two sequential reactions: 1) the conversion of pregnenolone and progesterone to their 17a-hydroxy derivatives by 17α-hydroxylase activity and 2) the subsequent formation of dehydroepiandrosterone (DHEA) and androstenedione, respectively, by C17,20 lyase activity. DHEA and androstenedione are androgens and are precursors of testosterone. Inhibition of CYP17 by Abiraterone can also result in increased mineralocorticoid production by the adrenals.

Androgen sensitive prostatic carcinoma responds to treatment that decreases androgen levels. Androgen deprivation therapies, such as treatment with GnRH agonists or orchiectomy, decrease androgen production in the testes but do not affect androgen production by the adrenals or in the tumor.

Abiraterone acetate decreased serum testosterone and other androgens in patients in the placebo-controlled phase 3 clinical trial. It is not necessary to monitor the effect of Abiraterone on serum testosterone levels.

Changes in serum prostate specific antigen (PSA) levels may be observed but have not been shown to correlate with clinical benefit in individual patients.

Following administration of Abiraterone acetate, the pharmacokinetics of Abiraterone and Abiraterone acetate have been studied in healthy subjects and in patients with metastatic castration-resistant prostate cancer (CRPC). In vivo, Abiraterone acetate is converted to Abiraterone. In clinical studies, Abiraterone acetate plasma concentrations were below detectable levels (<0.2 ng/mL) in >99% of the analyzed samples.

Following oral administration of Abiraterone acetate to patients with metastatic CRPC, the median time to reach maximum plasma Abiraterone concentrations is 2 hours. Abiraterone accumulation is observed at steady-state, with a 2-fold higher exposure (steady-state AUC) compared to a single 1,000 mg dose of Abiraterone acetate.

At the dose of 1,000 mg daily in patients with metastatic CRPC, steady-state values (mean±SD) of C. were 226±178 ng/mL and of AUC were 993±639 ng*hr/mL. No major deviation from dose proportionality was observed in the dose range of 250 mg to 1,000 mg. However, the exposure was not significantly increased when the dose was doubled from 1,000 to 2,000 mg (8% increase in the mean AUC).

Systemic exposure of Abiraterone is increased when Abiraterone acetate is administered with food. Abiraterone Cₘₐₓ and AUC₀₋₂₈ were approximately 7-and 5-fold higher, respectively, when Abiraterone acetate was administered with a low-fat meal (7% fat, 300 calories) and approximately 17-and 10-fold higher, respectively, when Abiraterone acetate was administered with a high-fat (57% fat, 825 calories) meal. Given the normal variation in the content and composition of meals, taking Zytiga® with meals has the potential to result in increased and highly variable exposures. Therefore, no food should be consumed for at least two hours before the dose of Zytiga® is taken and for at least one hour after the dose of Zytiga® is taken. The tablets should be swallowed whole with water. Abiraterone is highly bound (>99%) to the human plasma proteins, albumin and alpha-1 acid glycoprotein. The apparent steady-state volume of distribution (mean±SD) is 19,669±13,358 L. In vitro studies show that at clinically relevant concentrations, Abiraterone acetate and Abiraterone are not substrates of P-glycoprotein (P-gp) and that Abiraterone acetate is an inhibitor of P-gp. No studies have been conducted with other transporter proteins.

Following oral administration of ¹⁴C-abiraterone acetate as capsules, Abiraterone acetate is hydrolyzed to Abiraterone (active metabolite). The conversion is likely through esterase activity (the esterases have not been identified) and is not CYP mediated. The two main circulating metabolites of Abiraterone in human plasma are Abiraterone sulphate (inactive) and N-oxide Abiraterone sulphate (inactive), which account for about 43% of exposure each. CYP3A4 and SULT2A1 are the enzymes involved in the formation of N-oxide Abiraterone sulphate and SULT2A1 is involved in the formation of Abiraterone sulphate.

In patients with metastatic CRPC, the mean terminal half-life of Abiraterone in plasma (mean±SD) is 12±5 hours. Following oral administration of ¹⁴C-abiraterone acetate, approximately 88% of the radioactive dose is recovered in feces and approximately 5% in urine. The major compounds present in feces are unchanged Abiraterone acetate and Abiraterone (approximately 55% and 22% of the administered dose, respectively). The usual dose is 4 tablets (1,000 mg) taken together once a day. The tablets have to be swallowed with a glass of water on an empty stomach. The tablets have to be taken at least one hour before food, or at least 2 hours afterwards. Abiraterone has to be taken with a steroid called prednisolone to help reduce some of the side effects.

In clinical studies following the oral administration of Abiraterone acetate Abiraterone exhibited variable pharmacokinetics and an exceptionally large positive food effect. Abiraterone Cₘₐₓ and AUC₀₋₂₈ (exposure) were increased up to 17- and 10-fold higher, respectively, when a single dose of Abiraterone acetate was administered. In order to control Abiraterone plasma concentrations Zytiga® must be taken on an empty stomach. No food should be consumed for at least two hours before the dose of Zytiga® is taken and for at least one hour after the dose of Zytiga® is taken. The administered dose is also very large with 1 g taken once daily. Improving the oral bioavailability of the compound in the fasted state would therefore deliver two advantages: the abandoning of the requirement of taking the drug on an empty stomach and significant dose reduction. Based on the extent of the food effect of the currently used formula total elimination of it would allow 10-fold reduction of the dose.

### Darolutamide

Darolutamide is a new-generation nonsteroidal AR antagonist with a unique molecular structure. It comprises a mixture of two diastereomers, (S,R)-darolutamide (ORM-16497) and (S,S)-Darolutamide, which interconvert via the major metabolite keto-Darolutamide preferentially to (S,S)-Darolutamide; all three compounds show similar pharmacologic activity. Darulotamide's structure is:

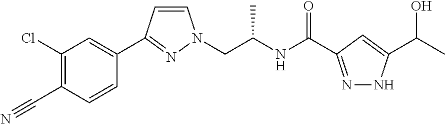 [SMILES: CC(O)c1cc(C(=O)N[C@@H](C)Cn2ccc(-c3ccc(C#N)c(Cl)c3)n2)n[nH]1]

In preclinical trials, Darolutamide demonstrated higher binding affinity compared with other AR antagonists (such as bicalutamide and enzalutamide), an antiproliferative effect and tumor growth inhibition in AR-overexpressing cells, and activity against AR mutants linked to drug resistance. In addition, darolutamide is different from other new-generation nonsteroidal AR antagonists with respect to its negligible bloodbrain barrier penetration. In early phase clinical trials with Western mCRPC patients, Darolutamide has shown a good safety profile and significant reductions in PSA levels.

Single-dose period pharmacokinetic (PK) assessment showed that the overall, median tₘₐₓ was 3-6 h for Darolutamide, demonstrating slow absorption; terminal half-life was in the range of 10-15 h. Cₘₐₓ and AUC_{(0-t last)} values were higher with 600 versus 300 mg under fasting and fed conditions. The CV for AUC_{(0-t last)} was higher for 300 mg (69.6%) versus 600 mg (41.4%) and higher than those under fed conditions (20.1 and 24.0%, respectively). Darolutamide achieved peak concentrations between 3 and 5 h postdose in the fasted state and between 3 and 8 h postdose in the fed state. In addition, tₘₐₓ was observed later under fed versus fasting conditions.

Under both fasted and fed conditions, dose-normalized values for Cₘₐₓ (Cₘₐₓ/D), AUC (AUC/D), and AUC_{(0-t last)} (AUC_{[0-t last]}/D) showed no relevant differences between the 300 and 600 mg doses, although AUC/D and Cₘₐₓ/D tended to be lower for Darolutamide 600 mg.

Administration of Darolutamide as a single oral dose under fed conditions demonstrated that bioavailability of Darolutamide was 2.5- and 2.8-fold higher (after 300 and 600 mg, respectively) versus Darolutamide given in fasting conditions. Similarly, the AUC(_{0-t last)} of Darolutamide for the fed state was 2.5-fold higher after 300 and 600 mg compared with the fasting state.

Darolutamide demonstrated a relatively flat PK profile at steady state that was most likely associated with the short dosing interval and its terminal half-life. On day 7 of the multiple-dose (md) period, Darolutamide Cₘₐₓ was reached 3-11 h after the dose taken with breakfast, with median t_{max.md} values of 4.98 and 5.48 h for 300 mg BID and 600 mg BID, respectively. Geometric mean C_{max,md} values for Darolutamide on day 7 were 4.60 and 5.80 μg/mL for 300 mg BID and 600 mg BID, respectively, which is approximately 1.8 and 1.7 times higher versus Cₘₐₓ values achieved after 300- and 600-mg single doses under fed conditions (2.59 and 3.50 μg/mL). Geometric mean AUC values for AUCₜₐᵤ(0-12)_{md} were 44.4 and 58.7 μg h/mL for Darolutamide 300 mg BID and 600 mg BID, corresponding to a 1.3-fold increase in exposure after multiple dosing with 600 mg BID versus 300 mg BID. Mean linearity factor (R_{LIN}) was comparable between the doses (0.910 for 300 mg BID, 0.961 for 600 mg BID). The dose-normalized parameter Cₘₐₓ/D_{md} and AUCₜₐᵤ(0-12)/D_{md} does not indicate any relevant differences between the 2 dose levels.

Median tₘₐₓ was shorter for diastereomer (S,R)-Darolutamide versus diastereomer (S,S)-Darolutamide at both Darolutamide dose levels when administered as single or multiple doses. Exposure to diastereomer (S,R)-Darolutamide was less versus diastereomer (S,S)-Darolutamide. The ratio of diastereomer (S,R)-Darolutamide AUC(0-tₗₐₛₜ) to diastereomer (S,S)-Darolutamide was approximately 1:4 (fasting) and 1:5 (fed) after a single dose of 300 mg, and approximately 1:7 (fasting) and 1:8 (fed) after a single dose of 600 mg.

The Cₘₐₓ of major metabolite keto-Darolutamide was higher compared with Darolutamide at both the 300- and 600-mg dose levels when administered as either single or multiple. Exposure to metabolite keto-Darolutamide was 1.28-fold (fasting) and 1.33-fold (fed) higher compared with Darolutamide after a single dose of 300 mg, and 1.44-fold (fasting) and 1.61-fold (fed) higher after a single dose of 600 mg. A similar food effect was observed for Cₘₐₓ. Food had no effect on tₘₐₓ (Nobuaki Matsubara et.al., Cancer Chemother Pharmacol., 2017; 80(6) pp1063-1072.)

In order to overcome the problems associated with prior conventional Abiraterone acetate and Darolutamide formulations and available drug delivery systems novel pharmaceutical composition of Abiraterone acetate and Darulotamide and pharmaceutically acceptable excipients characterized by instantaneous dissolution, reduced food effect which allows significant dose reduction and the abandoning of the requirement of taking the drug on an empty stomach was prepared.

A variety of strategies have been used to attempt to overcome these issues, see for example CN101768199A, CN102558275A, WO2014083512A1, WO2014145813A1, CN102321142A, WO2014102833A2, WO2014009436A1, WO2014145813A1, WO2014009434A1, WO2009009132A1, WO2013164473A1, WO1995011914A1, CA2513746A1, WO2010078300A1, WO2014100418A2 and WO2014009437A1.

### BRIEF DESCRIPTION OF THE INVENTION

Disclosed herein are pharmaceutical compositions with improved physicochemical characteristics and enhanced biological performance comprising
a) active compounds Abiraterone acetate and Darolutamide;
b) at least one primary pharmaceutical excipient chosen from polyethylene glycol glycerides composed of mono-, di- and triglycerides and mono- and diesters of polyethylene glycol, hydroxypropylcellulose, vinylpyrrolidone/vinyl acetate copolymer, polyethylene glycol, poly(2-ethyl-2-oxazoline), polyvinylpyrrolidone, block copolymers based on ethylene oxide and propylene oxide, poly(maleic acid/methyl vinyl ether), polyvinyl caprolactam-polyvinyl acetate-polyethylene glycol graft copolymer, ethylenediamine tetrakis(propoxylate-block-ethoxylate) tetrol; and
c) optionally, at least one secondary pharmaceutical excipient;
wherein said pharmaceutical composition possesses one or more of features i.-v.:
   i. it is instantaneously redispersible in physiological relevant media;
   ii. it has increased dissolution rate;
   iii. it is stable in solid form and in colloid solution and/or dispersion;
   iv. it has a PAMPA permeability of at least 0.5*10⁻⁶ cm/s when dispersed in distilled water;
   v. exhibits no positive food effect (fed/fasted ratio is under 1.25) which allows significant dose reduction and the abandoning of the requirement of taking the drugs on an empty stomach.

In an embodiment, said pharmaceutical composition possesses two or more of features i.-v.

In an embodiment, said pharmaceutical composition possesses three or more of features i.-v.

In an embodiment, the pharmaceutical composition comprises
a) said at least one primary pharmaceutical excipient is chosen from polyvinylpyrrolidone and vinylpyrrolidone/vinyl acetate copolymer; and
b) said at least one secondary pharmaceutical excipient is sodium lauryl sulfate.

In an embodiment, said pharmaceutical composition comprises
a) 0 to 40% by weight of Abiraterone acetate;
b) 0 to 40% by weight of Darolutamide;
c) 5 to 80% by weight of a primary pharmaceutical excipient is chosen from polyvinylpyrrolidone or vinylpyrrolidone/vinyl acetate copolymer; and
d) 0.1 to 50% by weight of a secondary pharmaceutical excipient that is sodium lauryl sulfate.

In an embodiment, Abiraterone acetate and Darolutamide in said pharmaceutical composition show amorphous character in X-ray powder diffraction studies.

Further disclosed herein is a process for the preparation of the pharmaceutical composition described herein, said process comprising the step of mixing a solution of the active agents and at least one primary pharmaceutical excipient and optionally one or more pharmaceutical excipient in a pharmaceutically acceptable solvent with an aqueous solution comprising optionally least one secondary pharmaceutical excipient.

In an embodiment, said process is performed in a continuous flow instrument.

In an embodiment, said pharmaceutically acceptable solvent is chosen from methanol, ethanol, isopropanol, n-propanol, acetone, acetonitrile, dimethyl-sulfoxide, tetrahydrofuran, or combinations thereof. In an embodiment, said pharmaceutically acceptable solvent is methanol.

In an embodiment, said pharmaceutically acceptable solvent and aqueous solution are miscible with each other and the aqueous solution comprises 0.1 to 99.9% weight of the final solution.

Disclosed herein are pharmaceutical dosage forms comprising the pharmaceutical composition, together with a pharmaceutically acceptable carrier.

In an embodiment, said pharmaceutical dosage form is suitable for oral, pulmonary, rectal, colonic, parenteral, intracisternal, intravaginal, intraperitoneal, ocular, otic, local, buccal, nasal, or topical administration.

In an embodiment, said pharmaceutical dosage form is suitable for oral administration.

Disclosed herein is the pharmaceutical composition for use in the manufacture of a medicament for the treatment of prostate cancer.

In an embodiment, the pharmaceutical composition is for use for the treatment of prostate cancer.

Provided herein is a method a treating prostate cancer comprising administration of the pharmaceutical composition as described herein.

### DESCRIPTION OF THE INVENTION

In an embodiment, the pharmaceutical composition comprises as active compound Abiraterone acetate and Darolutamide; and at least one primary pharmaceutical excipient chosen from polyethylene glycol glycerides composed of mono-, di- and triglycerides and mono- and diesters of polyethylene glycol, hydroxypropylcellulose, vinylpyrrolidone/vinyl acetate copolymer, polyethylene glycol, poly(2-ethyl-2-oxazoline), polyvinylpyrrolidone, block copolymers based on ethylene oxide and propylene oxide, poly(maleic acid/methyl vinyl ether), polyvinyl caprolactam-polyvinyl acetate-polyethylene glycol graft copolymer, ethylenediamine tetrakis(propoxylate-block-ethoxylate) tetrol said pharmaceutical composition characterized in that it possesses at least one of the following properties:
a) is instantaneously redispersible in physiological relevant media;
b) has increased dissolution rate;
c) is stable in solid form and in colloid solution and/or dispersion;
d) has a PAMPA permeability of at least 0.5*10⁻⁶ cm/s when dispersed in distilled water; and
e) exhibits no positive food effect which allows significant dose reduction and the abandoning of the requirement of taking the drug on an empty stomach.

In an embodiment, said composition shows X-ray amorphous character in the solid form.

In an embodiment, said pharmaceutical composition has increased in-vitro permeability and exhibits no positive food effect which allows significant dose reduction and the abandoning of the requirement of taking the drugs on an empty stomach.

It has been found that only selected combinations of primary pharmaceutical excipients and secondary pharmaceutical excipients result in a stable pharmaceutical composition having improved physicochemical characteristics and enhanced biological performance.

In an embodiment, said primary pharmaceutical excipient is chosen from polyethylene glycol glycerides composed of mono-, di- and triglycerides and mono- and diesters of polyethylene glycol, hydroxypropylcellulose, vinylpyrrolidone/vinyl acetate copolymer, polyethylene glycol, poly(2-ethyl-2-oxazoline), polyvinylpyrrolidone, block copolymers based on ethylene oxide and propylene oxide, poly(maleic acid/methyl vinyl ether), polyvinyl caprolactam-polyvinyl acetate-polyethylene glycol graft copolymer, ethylenediamine tetrakis(propoxylate-block-ethoxylate) tetrol.

In an embodiment, said primary pharmaceutical excipient is chosen from polyvinylpyrrolidone and vinylpyrrolidone/vinyl acetate copolymer.

In an embodiment, said primary pharmaceutical excipient is polyvinylpyrrolidone.

In an embodiment, said primary pharmaceutical excipient is a vinylpyrrolidone/vinyl acetate copolymer.

In an embodiment, said pharmaceutical composition further comprises sodium lauryl sulfate.

In an embodiment, said pharmaceutical composition comprises Abiraterone acetate and Darolutamide in amorphous forms.

In an embodiment, said pharmaceutical composition exhibits no positive food effect which allows significant dose reduction and the abandoning of the requirement of taking the drugs on an empty stomach.

In an embodiment, said pharmaceutical composition possesses at least two of the properties described in a)-e).

In an embodiment, said pharmaceutical composition possesses at least three of the properties described in a)-e).

In an embodiment, said complex has an increased dissolution rate.

Further disclosed herein is a pharmaceutical composition comprising an active compound Abiraterone acetate and Darolutamide, at least one primary pharmaceutical excipient chosen from polyethylene glycol glycerides composed of mono-, di- and triglycerides and mono- and diesters of polyethylene glycol, hydroxypropylcellulose, vinylpyrrolidone/vinyl acetate copolymer, polyethylene glycol, poly(2-ethyl-2-oxazoline), polyvinylpyrrolidone, block copolymers based on ethylene oxide and propylene oxide, poly(maleic acid/methyl vinyl ether), polyvinyl caprolactam-polyvinyl acetate-polyethylene glycol graft copolymer, ethylenediamine tetrakis(propoxylate-block-ethoxylate) tetrol.

In an embodiment, said primary pharmaceutical excipient agent is polyvinylpyrrolidone or vinylpyrrolidone/vinyl acetate copolymer.

In an embodiment, said pharmaceutical composition further comprises sodium lauryl sulfate.

In an embodiment, said pharmaceutical composition is obtained via a continuous flow mixing process.

In an embodiment, a pharmaceutical composition comprises primary pharmaceutical excipient which is polyvinylpyrrolidone or or vinylpyrrolidone/vinyl acetate copolymer and secondary pharmaceutical excipient which is sodium lauryl sulfate, in a total amount ranging from about 1.0 weight% to about 95.0 weight % based on the total weight of the pharmaceutical composition.

In an embodiment, a pharmaceutical composition comprises primary pharmaceutical excipient which is polyvinylpyrrolidone or or vinylpyrrolidone/vinyl acetate copolymer and secondary pharmaceutical excipient which is sodium lauryl sulfate, in a total amount ranging from about 5.0 weight% to about 95.0 weight % based on the total weight of the pharmaceutical composition.

In an embodiment, a pharmaceutical composition comprises primary pharmaceutical excipient which is polyvinylpyrrolidone or or vinylpyrrolidone/vinyl acetate copolymer and secondary pharmaceutical excipient which is sodium lauryl sulfate, in a total amount ranging from about 10.0 weight% to about 95.0 weight % based on the total weight of the pharmaceutical composition.

Further disclosed herein is a process for the preparation of the pharmaceutical composition, comprising the steps of mixing a solution of Abiraterone acetate and Darolutamide, and at least one primary pharmaceutical excipient and optionally one or more secondary pharmaceutical excipients in a pharmaceutically acceptable solvent with an aqueous solution comprising optionally least one secondary pharmaceutical excipient.

In an embodiment, said process is performed in a continuous flow instrument.

In an embodiment, said pharmaceutically acceptable solvent is chosen from methanol, ethanol, isopropanol, n-propanol, acetone, acetonitrile, dimethyl-sulfoxide, tetrahydrofuran, or combinations thereof.

In an embodiment, said pharmaceutically acceptable solvent is methanol.

In an embodiment, said pharmaceutically acceptable solvent and said aqueous solution are miscible with each other.

In an embodiment, said aqueous solution comprises 0.1 to 99.9% weight of the final solution.

In an embodiment, said aqueous solution comprises 50 to 90% weight of the final solution.

In an embodiment, said aqueous solution comprises 50 to 80% weight of the final solution.

In an embodiment, said aqueous solution comprises 50 to 70% weight of the final solution.

In an embodiment, said aqueous solution comprises 50 to 60% weight of the final solution.

In an embodiment, said aqueous solution comprises 50% weight of the final solution.

In an embodiment, said aqueous solution comprises 10 to 40% weight of the final solution.

In an embodiment, said aqueous solution comprises 10 to 30% weight of the final solution.

In an embodiment, said aqueous solution comprises 10 to 20% weight of the final solution.

In an embodiment, said aqueous solution comprises 10% weight of the final solution.

In an embodiment, a pharmaceutical dosage form comprises the pharmaceutical composition together with pharmaceutically acceptable carrier.

In an embodiment, said pharmaceutical dosage form is suitable for oral, pulmonary, rectal, colonic, parenteral, intracisternal, intravaginal, intraperitoneal, ocular, otic, local, buccal, nasal, or topical administration.

In an embodiment, said pharmaceutical dosage form is suitable for oral administration.

In an embodiment, said pharmaceutical composition is for use in the manufacture of a medicament for the treatment of prostate cancer.

In an embodiment, a method for reducing the therapeutically effective dosage of Abiraterone acetate and Darolutamide compared to commercially available dosage forms of Abiraterone acetate and Darolutamide comprises oral administration of a pharmaceutical composition as described herein.

Further disclosed herein is a stable complex comprising
a) 0 to 40% by weight of Abiraterone acetate;
b) 0 to 40% by weight of Darolutamide;
c) 5 to 80% by weight of a polyvinylpyrrolidone or or vinylpyrrolidone/vinyl acetate copolymer;
d) 0.1 to 50% by weight of sodium lauryl sulfate.

In an embodiment, said pharmaceutical composition exhibits no positive food effect based on in-vitro permeability studies.

In an embodiment, said pharmaceutical composition exhibits increased in-vitro permeability which allows significant dose reduction and the abandoning of the requirement of taking the drugs on an empty stomach.

In an embodiment, said pharmaceutical composition is instantaneously redispersable in physiological relevant media.

In an embodiment, said pharmaceutical composition is stable in solid form and in colloid solution and/or dispersion.

In an embodiment, said pharmaceutical composition shows X-ray amorphous character for Abiraterone acetate and Darolutamide in the solid form.

In an embodiment, said pharmaceutical composition has a PAMPA permeability of at least 0.5*10⁻⁶ cm/s when dispersed in distilled water.

In some embodiments, the pharmaceutical compositions may additionally include one or more pharmaceutically acceptable excipients, auxiliary materials, carriers, active agents or combinations thereof.

The pharmaceutical composition can be formulated: (a) for administration selected from the group consisting of oral, pulmonary, rectal, colonic, parenteral, intracisternal, intravaginal, intraperitoneal, ocular, otic, local, buccal, nasal, and topical administration; (b) into a dosage form selected from the group consisting of liquid dispersions, gels, aerosols, ointments, creams, lyophilized formulations, tablets, capsules; (c) into a dosage form selected from the group consisting of controlled release formulations, fast melt formulations, delayed release formulations, extended release formulations, pulsatile release formulations, and mixed immediate release and controlled release formulations; or (d) any combination of (a), (b), and (c).

The pharmaceutical compositions can be formulated by adding different types of pharmaceutically acceptable excipients for oral administration in solid, liquid, local (powders, ointments or drops), or topical administration, and the like.

In an embodiment, said pharmaceutical dosage form is a solid dosage form, although any pharmaceutically acceptable dosage form can be utilized.

Solid dosage forms for oral administration include, but are not limited to, capsules, tablets, pills, powders, and granules. In such solid dosage forms, the active agent is admixed with at least one of the following excipients: (a) one or more inert excipients (or carriers), such as sodium citrate or dicalcium phosphate; (b) fillers or extenders, such as starches, lactose, sucrose, glucose, mannitol, microcrystalline cellulose and silicic acid; (c) binders, such as cellulose derivatives, alginates, gelatin, polyvinylpyrrolidone, sucrose and acacia; (d) humectants, such as glycerol; (e) disintegrating agents, such as crospovidon, sodium starch glycolate, effervescent compositions, croscarmellose sodium calcium carbonate, potato or tapioca starch, alginic acid, certain complex silicates and sodium carbonate; (f) solution retarders, such as acrylates, cellulose derivatives, paraffin; (g) absorption accelerators, such as quaternary ammonium compounds; (h) wetting agents, such as polysorbates, cetyl alcohol and glycerol monostearate; (i) adsorbents, such as kaolin and bentonite; and (j) lubricants, such as talc, calcium stearate, magnesium stearate, solid polyethylene glycols, sodium lauryl sulfate, or mixtures thereof. For capsules, tablets, and pills, the dosage forms may also comprise buffering agents.

Besides such inert diluents, the composition can also include adjuvants, such as wetting agents, emulsifying and suspending agents, sweetening, flavoring, and perfuming agents.

Advantages of the pharmaceutical composition disclosed herein include, but are not limited to, (1) physical and chemical stability, (2) instantaneous redispersibility, (3) stability in colloid solution or dispersion in the therapeutic time window, (4) increased in-vitro permeability, (5) increased oral bioavailability in fasted state, (6) no positive food effect and (7) good processability.

Beneficial features of the pharmaceutical composition disclosed herein are as follows: the good/instantaneous redispersibility of solid pharmaceutical composition of Abiraterone acetate and Darolutamide in water, biologically relevant media, e.g. SGF, FessiF and FassiF media and gastro intestinal fluids and adequate stability in colloid solutions and/or dispersion in the therapeutic time window.

One of the characteristics of the pharmaceutical composition of Abiraterone acetate and Darolutamide of the pharmaceutical composition disclosed herein is the increased in-vitro permeability. In some embodiments, in-vitro permeability (PAMPA) of the pharmaceutical composition of Abiraterone acetate and Darolutamide is at least 0.5*10⁻⁶ cm/s.

Another characteristic of the pharmaceutical composition of Abiraterone acetate and Darolutamide of the pharmaceutical composition disclosed herein relates to the enhanced pharmacokinetic performance of the pharmaceutical composition of Abiraterone acetate and Darolutamide. It exhibits no positive food effect which allows significant dose reduction and the abandoning of the requirement of taking the drugs on an empty stomach.

### BRIEF DESCRIPTION OF THE DRAWINGS

FIG. 1 shows the pharmaceutical excipients screening for the selection of pharmaceutical composition having instantaneous redispersibility.

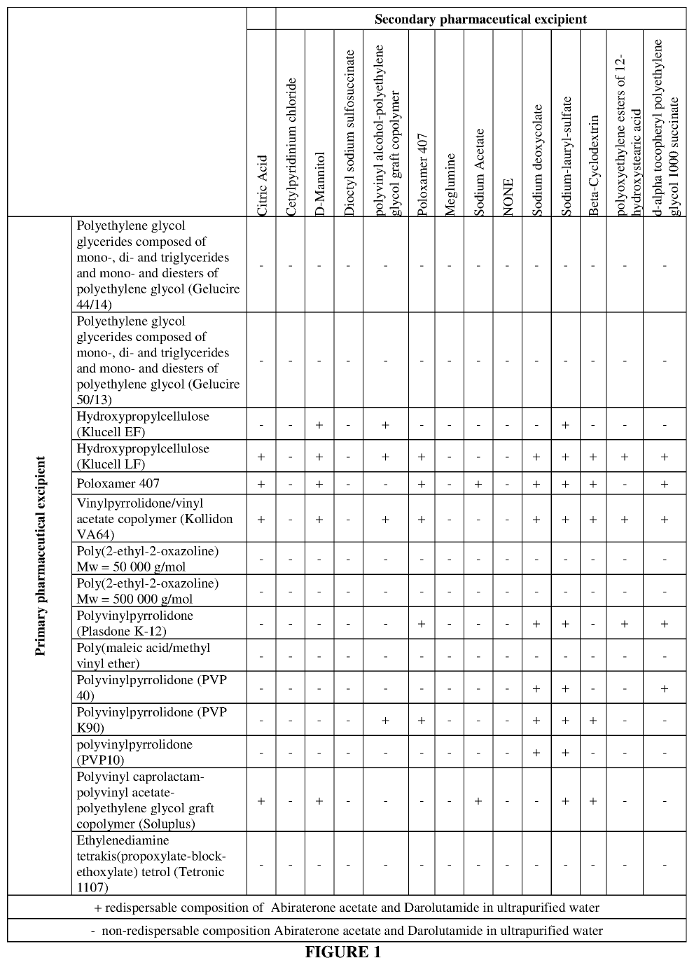

FIG. 2 shows comparative PAMPA assays of different pharmaceutical compositions of Abiraterone acetate and Darolutamide.

FIG. 3 shows comparative PAMPA assays of pharmaceutical composition of Abiraterone acetate and Darolutamide comprising primary and secondary pharmaceutical excipients in different ratios.

FIG. 4 shows the stability of the redispersed pharmaceutical composition at different time points.

FIG. 5 shows powder X-ray diffractograms of crystalline Abiraterone acetate, crystalline Darolutamide, SDS and pharmaceutical composition of Abiraterone acetate and Darolutamide formulation.

### EXAMPLES

Several pharmaceutical excipients and their combinations were tested in order to select the composition having instantaneous redispersibility as shown in FIG. 11. One of the examples that displayed an acceptable level of redispersibility was selected for further analysis.

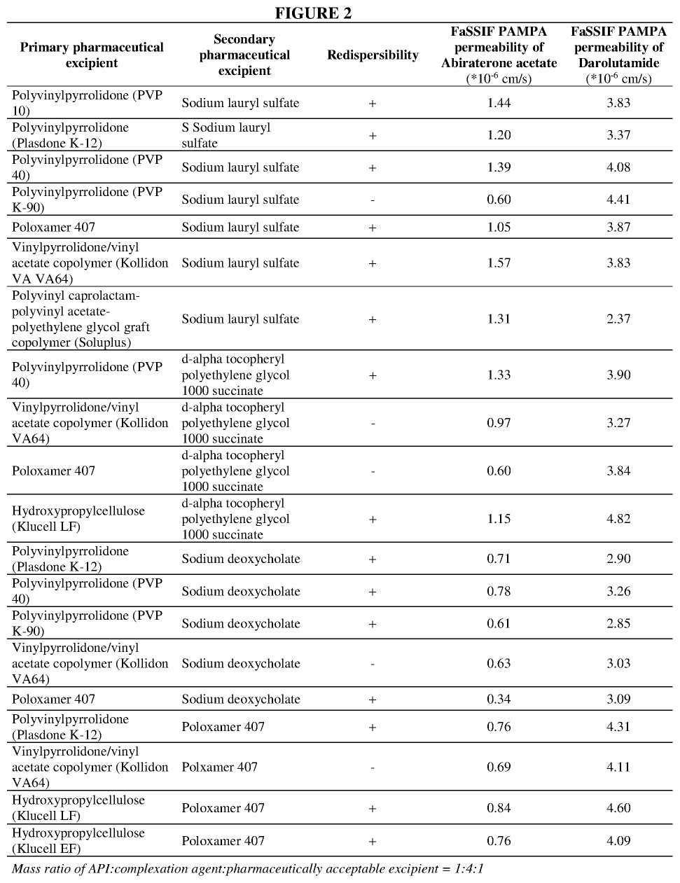

Parallel artificial membrane permeability assay (PAMPA) (in-vitro permeability) of the selected formulations was measured in order to select the pharmaceutical composition of Abiraterone acetate and Darolutamide having the best in-vitro performance (FIG. 2). PAMPA permeability measurements were performed as described by M. Kansi et al. (Journal of medicinal chemistry, 41, (1998) pp 1007) with modifications based on S. Bendels et al (Pharmaceutical research, 23 (2006) pp 2525). Permeability was measured in a 96-well plate assay across an artificial membrane composed of dodecane with 20% soy lecithin supported by a PVDF membrane (Millipore, USA). The receiver compartment was phosphate buffered saline (pH 7.0) supplemented with 1% sodium dodecyl sulfate. The assay was performed at room temperature; incubation time was 1-24 hours. The concentration in the receiver compartment was determined by UV-VIS spectrophotometry (Thermo Scientific Genesys S10).

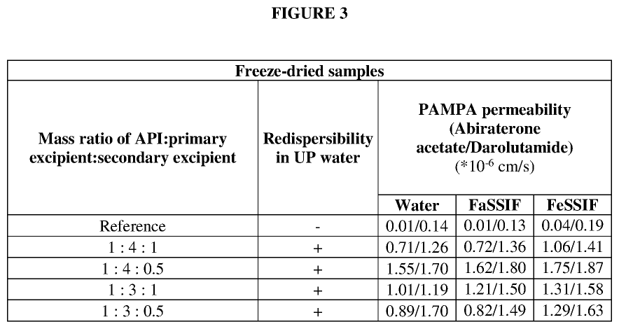

Polyvinylpyrrolidone (Plasdone K-12) as primary pharmaceutical excipient and sodium lauryl sulfate (SDS) as secondary pharmaceutical excipient were selected to prepare pharmaceutical composition of Abiraterone acetate and Darolutamide having improved material characteristics. The pharmaceutical composition having a ratio of Abiraterone acetate—Darolutamide, polyvinylpyrrolidone (Plasdone K-12) and sodium lauryl sulfate (SDS) that is 1:4:0.5 was found to have beneficial in-vitro properties (redispersibility profile, stability of the redispersed solution and PAMPA permeability) (FIG. 3).

The technological approach applied to the manufacture of the pharmaceutical composition of Abiraterone acetate and Darolutamide relied on freeze-drying of the solution mixture comprising Abiraterone acetate and Darolutamide and selected primary and secondary pharmaceutical excipients. The solution mixture was prepared by continuous flow mixing of two solutions. One of the solutions contained the Abiraterone acetate and Darolutamide and the primary pharmaceutical excipient(s) dissolved in methanol. The second solution was an aqueous solution comprising the secondary pharmaceutical excipient(s). The solution mixture was solidified right after the preparation using freeze-drying method.

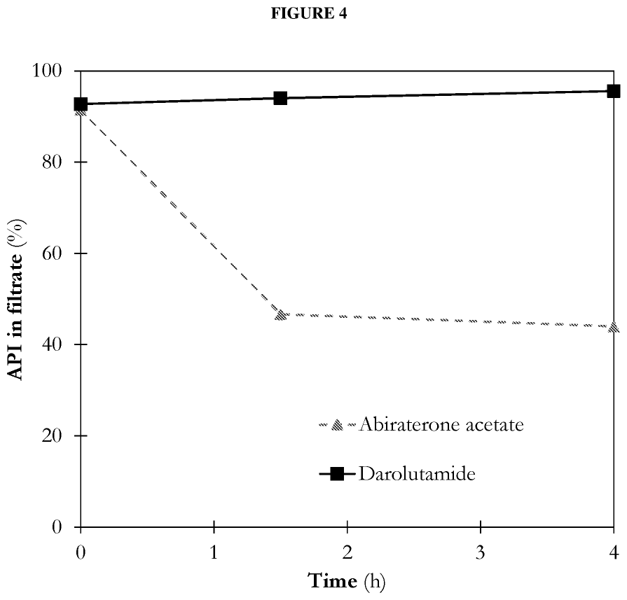

The stability of the redispersed freeze-dried samples was monitored. The solid pharmaceutical compositions of Abiraterone acetate and Darolutamide were redispersed in purified water using 4 mg/mL concentration for the Darolutamide and 1 mg/mL concentration for the Abiraterone acetate, respectively. The stability of redispersed pharmaceutical compositions was monitored by filtration test. The redispersed pharmaceutical compositions was filtered with 0.1 μm pore size filter at different time points. The Abiraterone acetate and Darolutamide contents of the filtrates were determined by HPLC (FIG. 4). The Darolutamide content of the filtrate was above 90%, while the Abiraterone acetate content was above 45% compared to the redispersed concentrations in the investigated time frame.

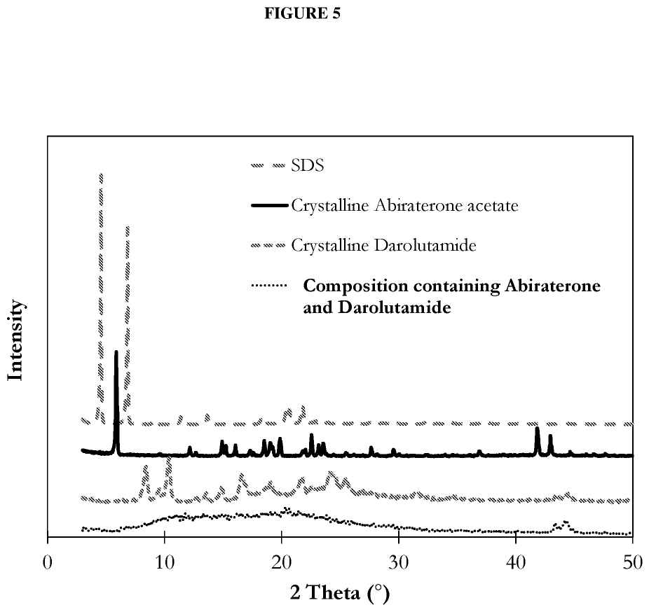

The structure of the pharmaceutical composition of Abiraterone acetate and Darolutamide was investigated by powder X-ray diffraction (XRD) analysis (Philips PW1050/1870 RTG powder-diffractometer). The measurements showed that both the Abiraterone acetate and Darolutamide were X-ray amorphous in the pharmaceutical composition (FIG. 5). Characteristic reflections on the diffractograms are be attributed to sample holder.

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A pharmaceutical composition with improved physicochemical characteristics and enhanced biological performance comprising:
a) active compounds Abiraterone acetate and Darolutamide;
b) at least one primary pharmaceutical excipient chosen from polyethylene glycol glycerides composed of mono-, di- and triglycerides and mono- and diesters of polyethylene glycol, hydroxypropylcellulose, vinylpyrrolidone/vinyl acetate copolymer, polyethylene glycol, poly(2-ethyl-2-oxazoline), polyvinylpyrrolidone, block copolymers based on ethylene oxide and propylene oxide, poly(maleic acid/methyl vinyl ether), polyvinyl caprolactam-polyvinyl acetate-polyethylene glycol graft copolymer, ethylenediamine tetrakis(propoxylate-block-ethoxylate) tetrol; and
c) optionally, at least one secondary pharmaceutical excipient;
wherein said pharmaceutical composition possesses one or more of features i.-v.:
i. it is instantaneously redispersible in physiological relevant media;
ii. it has increased dissolution rate;
iii. it is stable in solid form and in colloid solution and/or dispersion;
iv. it has a PAMPA permeability of at least 0.5*10⁻⁶ cm/s when dispersed in distilled water;
v. exhibits no positive food effect (fed/fasted ratio is under 1.25) which allows significant dose reduction and the abandoning of the requirement of taking the drugs on an empty stomach.

2. The pharmaceutical composition as recited in claim 1, wherein said pharmaceutical composition possesses two or more of features i.-v.

3. The pharmaceutical composition as recited in claim 2, wherein said pharmaceutical composition possesses three or more of features i.-v.

4. The pharmaceutical composition as recited in claim 1, wherein said composition comprises
a) said at least one primary pharmaceutical excipient is chosen from polyvinylpyrrolidone and vinylpyrrolidone/vinyl acetate copolymer; and
b) said at least one secondary pharmaceutical excipient is sodium lauryl sulfate.

5. The pharmaceutical composition as recited in claim 1, wherein said pharmaceutical composition comprises
a) 0 to 40% by weight of Abiraterone acetate;
b) 0 to 40% by weight of Darolutamide;
c) 5 to 80% by weight of a primary pharmaceutical excipient is chosen from polyvinylpyrrolidone or vinylpyrrolidone/vinyl acetate copolymer; and
d) 0.1 to 50% by weight of a secondary pharmaceutical excipient that is sodium lauryl sulfate.

6. The pharmaceutical composition as recited in claim 4 wherein Abiraterone acetate and Darolutamide in said pharmaceutical composition show amorphous character in X-ray powder diffraction studies.

7. A process for the preparation of a pharmaceutical composition as recited in claims 1 to 4, said process comprising the step of mixing a solution of the active agents and at least one primary pharmaceutical excipient and optionally one or more pharmaceutical excipient in a pharmaceutically acceptable solvent with an aqueous solution comprising optionally least one secondary pharmaceutical excipient.

8. The process as claimed in claim 7, wherein said process is performed in a continuous flow instrument.

9. The process as recited in claim 7, wherein said pharmaceutically acceptable solvent is chosen from methanol, ethanol, isopropanol, n-propanol, acetone, acetonitrile, dimethyl-sulfoxide, tetrahydrofuran, or combinations thereof.

10. The process as recited in claim 7, wherein said pharmaceutically acceptable solvent and aqueous solution are miscible with each other and the aqueous solution comprises 0.1 to 99.9% weight of the final solution.

11. A pharmaceutical dosage form comprising the pharmaceutical composition as recited in claim 1, together with a pharmaceutically acceptable carrier.

12. The pharmaceutical dosage form as recited in claim 11, wherein said pharmaceutical dosage form is suitable for oral, pulmonary, rectal, colonic, parenteral, intracisternal, intravaginal, intraperitoneal, ocular, otic, local, buccal, nasal, or topical administration.

13. The pharmaceutical dosage form as recited in claim 12, wherein said pharmaceutical dosage form is suitable for oral administration.

14. The pharmaceutical composition as recited in claim 1 for use in the manufacture of a medicament for the treatment of prostate cancer.

15. The pharmaceutical composition as recited in claim 1 for use for the treatment of prostate cancer.

16. A method a treating prostate cancer comprising administration of the pharmaceutical composition as recited in claim 1.
